(19) United States
(12) Patent Application Publication
(10) Pub. No.: US 2005/0033445 A1
(43) Pub. Date: Feb. 10, 2005

(54) Hip joint prosthesis

(76) Inventor: Thomas Siebel, Saarbrucken (DE)

Correspondence Address: Friedrich Kueffner, Suite 910, 317 Madison Avenue, New York, NY, 10017, US

(21) Appl. No.: 10/704,064

(22) Filed: Nov. 7, 2003

(30) Foreign Application Priority Data
Aug. 5, 2003 (DE) 103 36 541.9
Aug. 18, 2003 (DE) 103 38 420.0

(51) Int. Cl. A61F 2/32

(52) U.S. Cl. 623/22.15

(57) ABSTRACT

A hip joint prosthesis has a socket part and a head rotatably arranged within the socket part. The socket part has an outer side facing away from the head and the outer side has a gliding surface configured to rotatably support the socket part in a natural hip socket. The socket part is polished on the outer side for forming the gliding surface. A stop is provided on the head or on the hipbone for limiting rotation of the socket part. The socket part has an angled edge portion that is arranged in an edge depression of the hip socket and strikes on the hipbone. The rotation of the socket part is limited in this way.

### BACKGROUND OF THE INVENTION

1. Field of the Invention

The invention relates to a hip joint prosthesis comprising a socket part and a head or ball rotatably supported in the socket part.

2. Description of the Related Art

Hip joint prostheses that are known in practice have a socket part or cup provided for being pressed into the optionally widened natural hip socket. The surface of the socket part on the side facing away from the head or ball is provided with a rough coating that stimulates ingrowth of the bone tissue. When pressing the socket part into place, permanent deformations of the spherical surface of the socket part receiving the head can occur; this impairs the proper function of the hip joint prosthesis.

### SUMMARY OF THE INVENTION

It is an object of the present invention to provide a new hip joint prosthesis of the aforementioned kind whose socket part can be implanted in a load-free way.

In accordance with the present invention, this is achieved in that the socket part on the side facing away from the head has a gliding surface for rotatably supporting the socket part in the natural hip socket.

According to the invention, no fixed anchoring of the socket part in the hipbone takes place. The socket part that is polished on both sides to provide gliding surfaces can be inserted into the natural hip socket without there being the risk of deformation. By providing an outer gliding surface on the socket part, loading of the cartilage layer in the hip socket is prevented.

In a particularly preferred embodiment of the invention, a stop limiting the rotation of the socket part in the natural hip socket is formed; the stop prevents that the socket part will project too far from the hip socket and will damage with its edge the surrounding tissue.

Such a stop for limiting the rotation of the socket part can be provided on the head and/or on the hipbone.

In the latter case, the socket part has preferably a flange-like angled edge portion for striking against the hipbone at the edge of the hip socket. In particular, this angled edge portion can be arranged within an edge depression formed on the hip socket. The shoulder that is formed by this edge depression then forms the stop for limiting the rotation of the socket part in the hip socket.

Preferably, the socket part extends across such an angle that in any rotational position, in which the angled edge portion strikes against the shoulder, the angled edge portion remains completely within the edge depression.

In a further embodiment, the stop limiting the rotation of the socket part can be formed by a widened, in particular, angular, portion of the outer surface of the spherical joint cap of the head. For this purpose, the spherical surface of the joint cap can pass into a cylindrical or conical surface.

In an alternative embodiment, the stop for limiting the rotation of the socket part is formed by the rim of a cutout in the outer surface of the spherical joint cap of the head against which a projection projecting from the inner surface of the socket part strikes.

In a reverse arrangement, such a stop can also be formed by a projection projecting from the outer surface of a spherical joint cap of the head against which the rim of a cutout provided on the inner surface of the socket part will strike.

### BRIEF DESCRIPTION OF THE DRAWING

In the drawing:

FIG. 1 shows a first embodiment of a hip joint prosthesis according to the invention;

FIG. 2 shows a milling tool usable for implanting the hip joint prosthesis of FIG. 1;

FIG. 3 shows a second embodiment of a hip joint prosthesis according to the invention;

FIG. 4 shows a third embodiment of a hip joint prosthesis according to the invention;

FIG. 5 shows a fourth embodiment of a hip joint prosthesis according to the invention;

FIG. 6 shows a fifth embodiment of a hip joint prosthesis according to the invention;

FIG. 7 shows a sixth embodiment of a hip joint prosthesis according to the invention;

FIG. 8 shows a seventh embodiment of a hip joint prosthesis according to the invention; and

FIG. 9 shows a eighth embodiment of a hip joint prosthesis according to the invention.

### DESCRIPTION OF THE PREFERRED EMBODIMENTS

The hip joint illustrated in FIG. 1 has a socket part 1 and a head 2. The socket part can be inserted into the natural hip socket 4 formed in the hipbone 3. The head 2 has a spherical joint cap 5 to be placed onto a prepared femoral head and a central pin 6 connected to the spherical cap for anchoring the head in the bone of the femoral head.

The spherical outer surfaces 7 of the spherical cap 5 is polished for increasing the gliding properties. When the hip joint prosthesis is implanted, the outer surface 7 rests against a spherical polished inner surface 8 of the socket part 1.

The spherical outer side 9 of the socket part 1 facing away from the head is also polished so that a gliding surface is formed with which the implanted socket part 1 rests against the cartilage layer 10 in the hip socket 4.

As illustrated in FIG. 1, the socket part 1 is provided with a flange-like angled edge portion 11. The angled edge portion 11 is received in an edge depression 12 of the hip socket wherein the edge depression 12 in the hip socket forms an annular shoulder 13. When the socket part 1 is implanted, the angled edge portion 11 is, for example, in a position indicated at 14 in dashed lines.

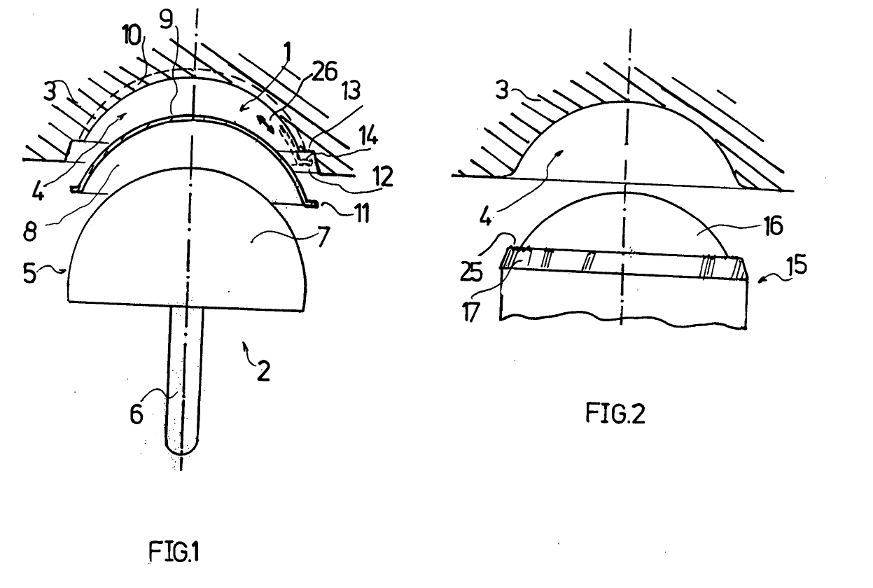

For forming the edge depression 12, a milling head 15 illustrated in FIG. 2 is provided that has a spherical end face 16 and a milling ring 17 provided with cutting elements. By means of the milling ring 17, a shoulder 25 is formed that matches the annular shoulder 13.

The socket part 1 of the implanted prosthesis can glide on the cartilage layer 10 of the hip socket 4 and is thus rotatable according to the double arrows 26 within the hip socket; this movability is limited by a stop formed by the annular shoulder 13 for the angled edge portion 11. This prevents that the socket part 1 that can glide and thus rotationally move on the cartilage layer 10 can move out of the hip socket 4 and can cause injuries in the surroundings of the hip joint which would cause ailments and, in the end, would require another surgical procedure.

In the illustrated embodiment, the socket part, in any rotational position in which it rests with its angled edge portion 11 against the annular shoulder 13, does not project at any location from the hip socket 4 that is provided with the depression, i.e., the location of the angled edge portion 11 diametrically opposed to the stop location remains within the edge depression 12. In this way, injuries in the surroundings of the implant can be substantially precluded.

In the following Figures, same parts or parts that act in the same way are identified with the same reference numerals as in FIGS. 1 and 2, wherein the corresponding reference numerals have letters a, b, etc. attached thereto.

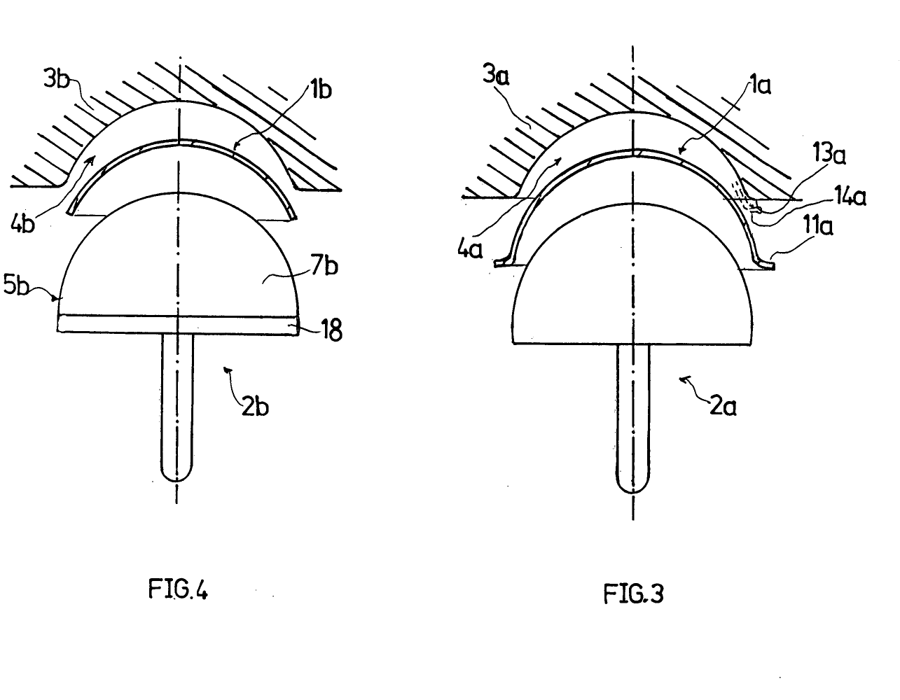

The socket part 1a of the hip joint prosthesis illustrated in FIG. 3 extends across an angle of approximately 180°. A flange-like angled edge portion 11a of the socket part inserted into the hip socket 4a projects in this embodiment from the hip socket and is, for example, in the position 14a illustrated by the dashed lines. In this position, the angled edge portion is spaced by approximately 2 mm from the stop shoulder 13a formed by the hipbone.

In the embodiment of FIG. 3, the movement range of the socket part 1a of the implanted hip joint is also limited by the angled edge portion 11a striking against the edge surface 13a and injuries of the surrounding tissue by an excessive projection of the socket part from the hip socket is precluded.

In the embodiment of FIG. 4, a stop for the socket part 1b is formed by an annular widened portion 18 on the spherical joint cap 5b of a head 2b. In this embodiment, the annular widened portion 18 has a cylindrical circumferential surface 19 and adjoins the spherical polished outer surface 7b of the spherical joint cap 5b. The socket part 1b that is glidingly movable with in the hip socket 4b can move on the outer surface 7b maximally to the annular widened portion 18. A complete escape of the socket part 1b, extending across an angle of approximately 150°, out of the hip socket 4b is prevented in this way.

In the embodiment according to FIG. 5, the spherical outer surface 7c of a spherical joint cap 5c is provided with a cutout 19 having a rim 20 providing a stop for the central projection 21 projecting from the inner surface 8c of a socket part 1c.

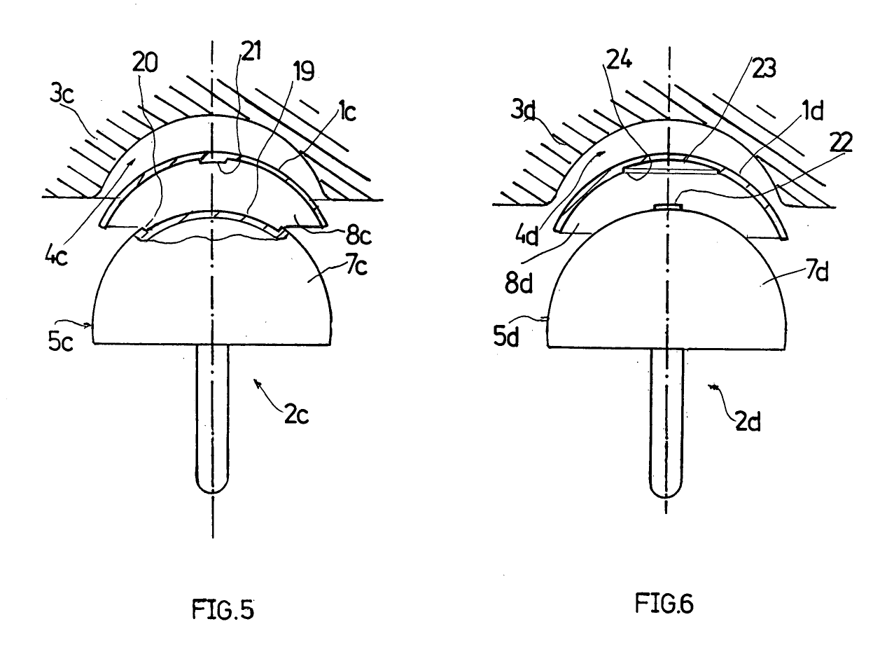

In the embodiment according to FIG. 6, a projection 22 projects centrally from the polished outer surface 7d of a cap part 5d. The projection 22 engages a cutout 23 in the inner surface 8d of a socket part 1d and strikes against the rim 24.

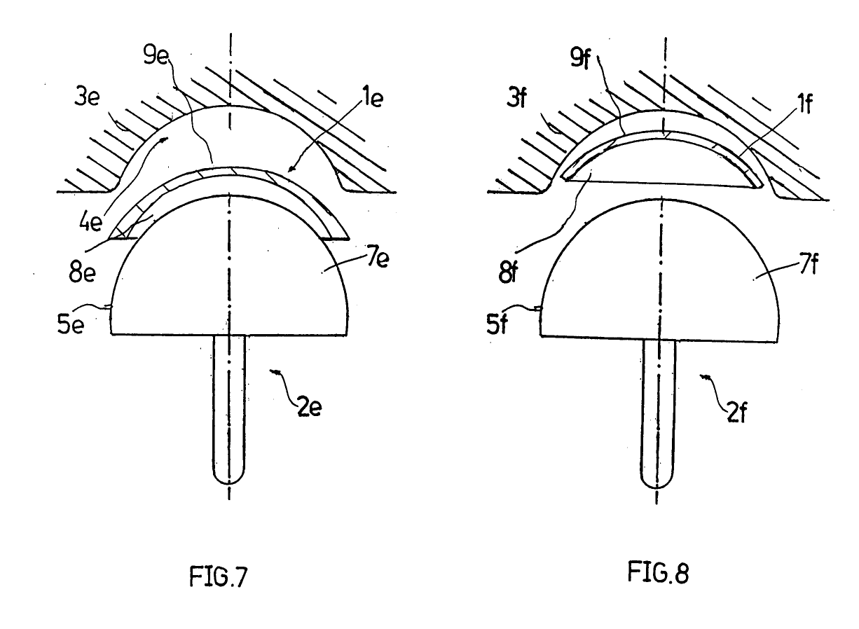

In the embodiment according to FIG. 7, an inner surface 8e of a socket part 1e is arranged eccentrically relative to the outer side 9e wherein the center point of the sphere of the inner surface 8e relative to the center point of the sphere of the outer surface 9e is moved toward the hip socket 4e. Eccentricity can also be provided in the reverse direction, i.e., the socket part can be thicker at the bottom than at the edge, as illustrated in FIG. 9. In both situations, the shape of the socket part ensures that escape of the socket part from the hip socket is made difficult. In addition, stops like the ones illustrated in the above Figures could also be provided.

FIG. 8 shows an embodiment without any measure for limiting the movability of a socket part 1f in a hip socket 3f. An inner surface 8f is concentric to the outer surface 9f of a socket part 1f. The risk of escape of the socket part from the hip socket is reduced in that it extends across a relatively small angle and the edge of the socket part is recessed from the edge of the hip socket. Still, the angle could be greater and could be up to 180°.

While specific embodiments of the invention have been shown and described in detail to illustrate the inventive principles, it will be understood that the invention may be embodied otherwise without departing from such principles.

1. A hip joint prosthesis comprising:
a socket part and a head rotatably arranged within the socket part;
wherein the socket part has an outer side facing away from the head and wherein the outer side has a gliding surface configured to rotatably support the socket part in a natural hip socket.

2. The hip joint prosthesis according to claim 1, wherein the socket part is polished on the outer side for forming the gliding surface.

3. The hip joint prosthesis according to claim 1, wherein means are provided for limiting rotation of the socket part in the natural hip socket.

4. The hip joint prosthesis according to claim 3, wherein a stop is provided for limiting rotation of the socket part.

5. The hip joint prosthesis according to claim 4, wherein the stop is provided on at least one of the head and a hipbone.

6. The hip joint prosthesis according to claim 5, wherein the socket part has a flange-shaped angled edge portion for striking against the hipbone at an edge of the natural hip socket.

7. The hip joint prosthesis according to claim 6, wherein the angled edge portion is configured to rest against a shoulder formed as an edge depression on the edge of the hip socket.

8. The hip joint prosthesis according to claim 7, wherein the angled edge portion remains completely within the edge depression in any rotational position in which rotational position the angled edge portion rests against the shoulder.

9. The hip joint prosthesis according to claim 4, wherein the head comprises a spherical joint cap, wherein the stop limiting the rotation of the socket part is formed by a widened portion of an outer surface of the spherical joint cap.

10. The hip joint prosthesis according to claim 9, wherein the widened portion is annular.

11. The hip joint prosthesis according to claim 4, wherein the head comprises a spherical joint cap having an outer surface, wherein the stop limiting the rotation of the socket part is formed by a rim of a recess provided in the outer surface of the spherical joint cap, wherein the socket part has an inner surface provided with a projection and wherein the projection strikes against the rim.

12. The hip joint prosthesis according to claim 4, wherein the head comprises a spherical joint cap having an outer surface, wherein the stop for limiting the rotation of the socket part is formed by a projection projecting form the outer surface of the spherical joint cap, wherein the socket part has an inner surface provided with a cutout having a rim, wherein the projection strikes against the rim.

13. The hip joint prosthesis according to claim 1, wherein the socket part has an inner surface and wherein the inner surface is arranged eccentrically relative to the outer side of the socket part.
